(19) United States
(12) Patent Application Publication
(10) Pub. No.: US 2012/0101262 A1
(43) Pub. Date: Apr. 26, 2012

(54) PROTEIN PURIFICATION BY CAPRYLIC ACID (OCTANOIC ACID) PRECIPITATION

(75) Inventors: Alahari Arunakumari, Pennington, NJ (US); Jue Wang, Bridgewater, NJ (US); Timothy Kyle Diehl, Marietta, NY (US)

(73) Assignee: BRISTOL-MYERS SQUIBB COMPANY, Princeton, NJ (US)

(21) Appl. No.: 13/380,609

(22) PCT Filed: Jun. 24, 2010
(86) PCT No.: PCT/US2010/039771

§ 371 Date: Dec. 23, 2011

Related U.S. Application Data
(60) Provisional application No. 61/220,549, filed on Jun. 25, 2009.

(51) Int. Cl. C07K 16/00; C07K 1/30; C07K 14/00

(52) U.S. Cl. 530/387.3; 530/418; 530/389.1; 530/388.1

(57) ABSTRACT

The invention provides methods for a purifying protein of interest from a mixture comprising the protein of interest and one or more contaminants, including host cell DNA and proteins, by precipitation of the contaminants with caprylic acid. Such methods are particularly useful for purifying antibodies from cell cultures. Moreover, mixtures that have been depleted of contaminants using the methods of the invention can be used directly in downstream chromatography applications (e.g., ion exchange chromatography) without any further purification. These methods lead to manufacturing processes with a minimum number of unit operations and reduce the resource requirements, and thus positively influence the cost of goods for therapeutic protein production.

### CROSS REFERENCE TO RELATED APPLICATION

This application claims the benefit of U.S. Provisional Application No. 61/220,549, filed Jun. 25, 2009, which is hereby incorporated by reference in its entirety.

### BACKGROUND OF THE INVENTION

The large-scale, economic purification of proteins is an increasingly important problem for the biopharmaceutical industry. Therapeutic proteins are typically produced using prokaryotic or eukaryotic cell lines that are engineered to express the protein of interest from a recombinant plasmid containing the gene encoding the protein. Separation of the desired protein from the mixture of components fed to the cells and cellular by-products to an adequate purity, e.g., sufficient for use as a human therapeutic, poses a formidable challenge to biologics manufacturers. For example, in therapeutic antibody purification, the current industry-standard chromatography capture resin, Protein A, is expensive, has a relatively low throughput, and has limited life cycles.

Accordingly there is a need in the art for alternative protein purification methods that can be used to expedite the large-scale processing of protein-based therapeutics, such as antibodies especially due to escalating high titers from cell culture.

### SUMMARY OF THE INVENTION

The present invention is based on the discovery that therapeutic proteins, particularly antibodies, can be efficiently purified (i.e., separated from a mixture comprising the protein and at least one contaminant) by precipitation of the contaminants with caprylic acid. When used to purify an antibody, for example, such methods result in the isolation of the antibody from host cell contaminants, such as host cell proteins and nucleic acid (e.g., deoxyribonucleotides (DNA)). The methods of the invention are particularly advantageous in that they can be performed directly on cell cultures, or lysates thereof, in a bioreactor without first removing the cells or cellular debris. Moreover, mixtures that have been depleted of contaminants (e.g., host cell contaminants) using the methods of the invention can be used directly in downstream chromatography applications (e.g., ion exchange chromatography) without any further purification.

Accordingly, in one aspect, the invention provides a method of purifying a protein (e.g., an antibody) from a mixture (e.g., cell culture, cell lysate or clarified bulk) comprising one or more contaminants, including host cell contaminants (e.g., host cell proteins or nucleic acids). The method generally comprises (a) adding caprylic acid to the mixture to form a contaminant precipitate; and (b) separating the contaminant precipitate from the cell culture, thereby purifying the protein of interest. Such contaminant precipitates can be separated from the mixture using any art recognized means, such as centrifugation, depth filtration or tangential flow filtration. In certain embodiments, caprylic acid treatment of a cell culture, cell lysate or clarified bulk may result in the removal of at least 60% (e.g., 60, 65, 70, 75, 80, 85, 90, 91, 92, 93, 94, 95, 96, 97, 98 or 99%) of the contaminants. For example, the level of host cell protein in the mixture may be reduced to less than about 10000, 5000, 1000, 500, 100, 90, 80, 70, 60, 50, 40, 30, 20 or 10 ng/mg and the level of nucleic acid in the mixture may be reduced to less than about 500, 100, 50, 10, 5, 4, 3, 2, 1 or 0.5 pg/mg. In certain aspects, over 60% (e.g., 65, 70, 75, 80, 85, 90, 91, 92, 93, 94, 95, 96, 97, 98 or 99%) of the protein of interest remains in the mixture after separation from the contaminant precipitate.

In certain embodiments, the methods of the invention further comprise the step of subjecting the purified protein to chromatography selected from the group consisting of ion exchange, hydrophobic interaction, affinity, mimetic, and mixed mode.

In certain embodiments, the caprylic acid is added directly to a bioreactor containing a cell culture, such as a mammalian cell culture (e.g., a Chinese Hamster Ovary (CHO) cell culture). The cells in the cell culture can be intact, or lysed prior to the addition of caprylic acid. Cell cultures can also be cleared of cells or cellular debris (e.g., to produce a clarified bulk) prior to the addition of caprylic acid. The final concentration of caprylic acid added to the mixture is between about 0.05 and 5% (v/v).

In certain embodiments, the pH of the mixture is altered. Such pH alteration can occur before or after the addition of caprylic acid. In a particular embodiment, the pH is altered to be between about 3 and 8. In another particular embodiment, the pH of the mixture is altered to be less than 5.

In certain embodiments, the contaminant precipitate is allowed to form for between about 30 to 120 minutes after addition of the caprylic acid (e.g., between about 30 to 60 minutes).

In another aspect, the invention provides a method of removing contaminants (e.g., host cell proteins and nucleic acids) from a mixture (e.g., a cell culture, cell lysate and clarified bulk). The method generally comprises adjusting the pH of the mixture to less than 5, adding caprylic acid to the mixture to precipitate the contaminants, thereby removing them from the mixture. In a particular embodiment, the mixture is obtained from a cell culture or cell lysate, with the proviso that the method is performed prior to obtaining a clarified bulk. In another particular embodiment, the mixture is contained in a bioreactor. In another particular embodiment, the mixture is a cell culture supernatant.

The methods of the invention can be used to purify any type of protein from a mixture. In a particular embodiment, the methods are employed to purify an antibody, such as a monoclonal antibody (e.g., a human, humanized or chimeric monoclonal antibody) or a fragment thereof, from cell culture (e.g., a mammalian, bacterial, plant or fungal cell culture), cell lysate, clarified bulk (e.g., clarified cell culture supernatant), or transgenic plant or animal derived protein mixture or extract. In certain embodiments, the methods comprise effectively removing contaminants from a mixture (e.g., a cell culture, cell lysate or clarified bulk) which contains a high concentration of a protein of interest (e.g., an antibody). For example, the concentration of a protein of interest may range from about 0.5 to about 50 mg/ml (e.g., 0.5, 1, 5, 10, 15, 20, 25, 30, 35, 40, 45 or 50 mg/ml).

### BRIEF DESCRIPTION OF THE DRAWINGS

FIG. 1 shows a schematic representation of a method for the removal of contaminants from a mixture using caprylic acid precipitation.

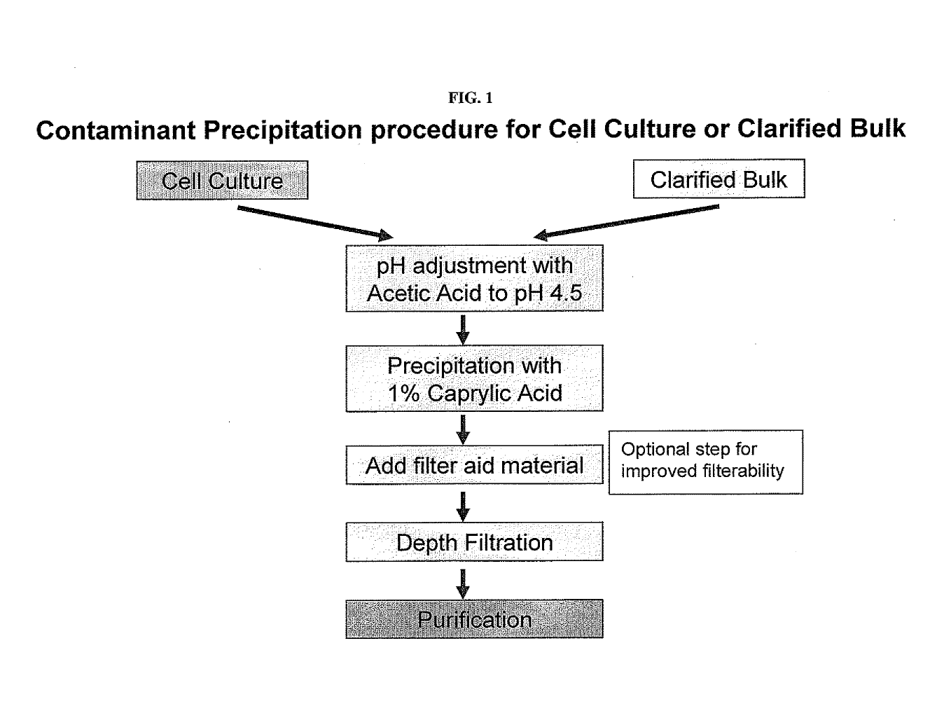

FIG. 2 shows a schematic representation of a method for the removal of contaminants from a mixture using caprylic acid precipitation coupled with several alternative downstream chromatography steps.

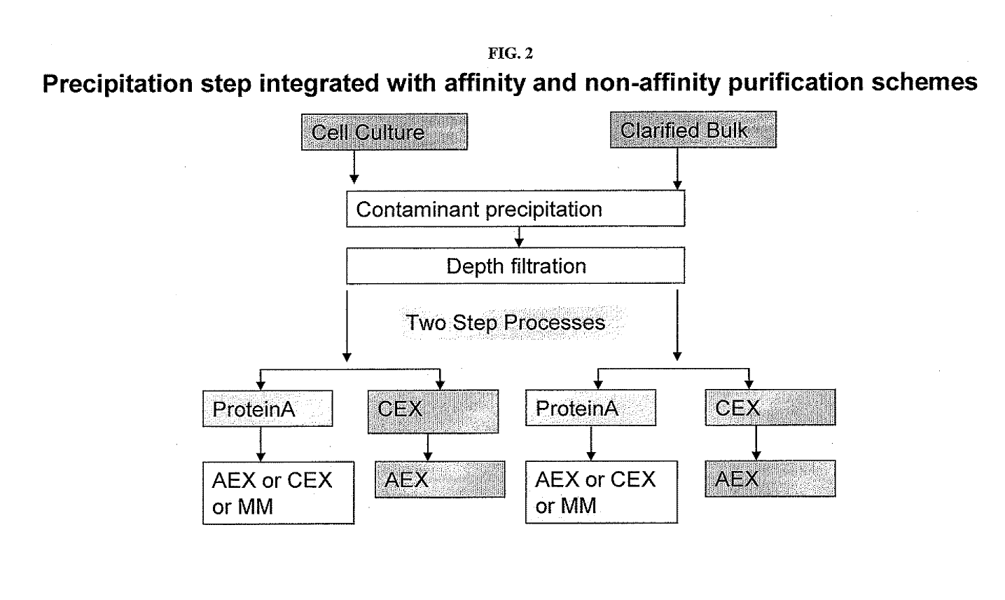

FIG. 3 shows a schematic representation of a typical human antibody purification scheme. The dotted rectangle indicates the steps that can be replaced by a single caprylic acid precipitation step.

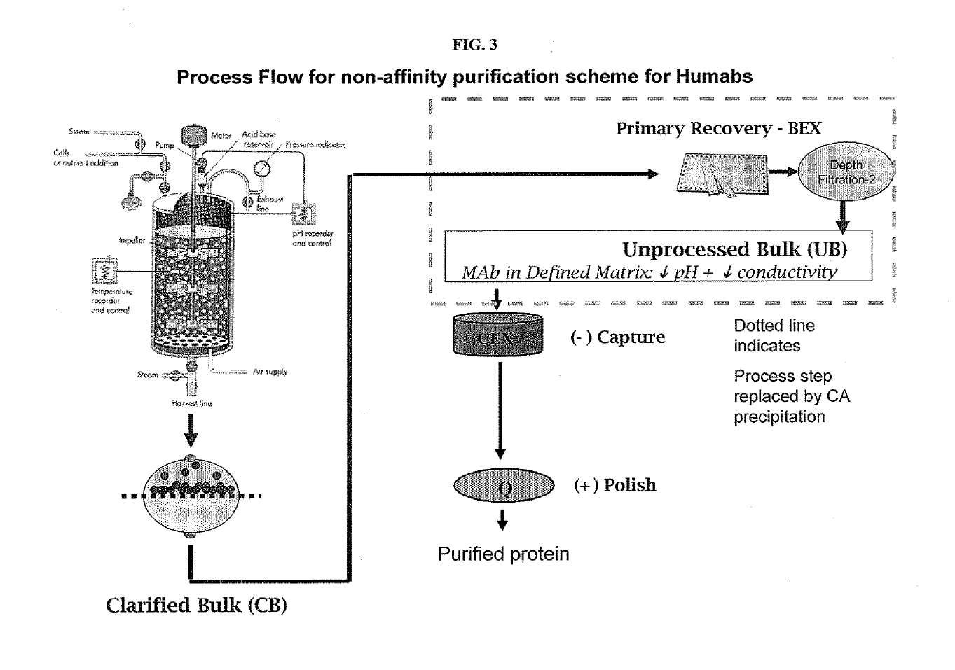

FIG. 4 shows a schematic representation of a caprylic acid precipitation purification scheme for a human antibody.

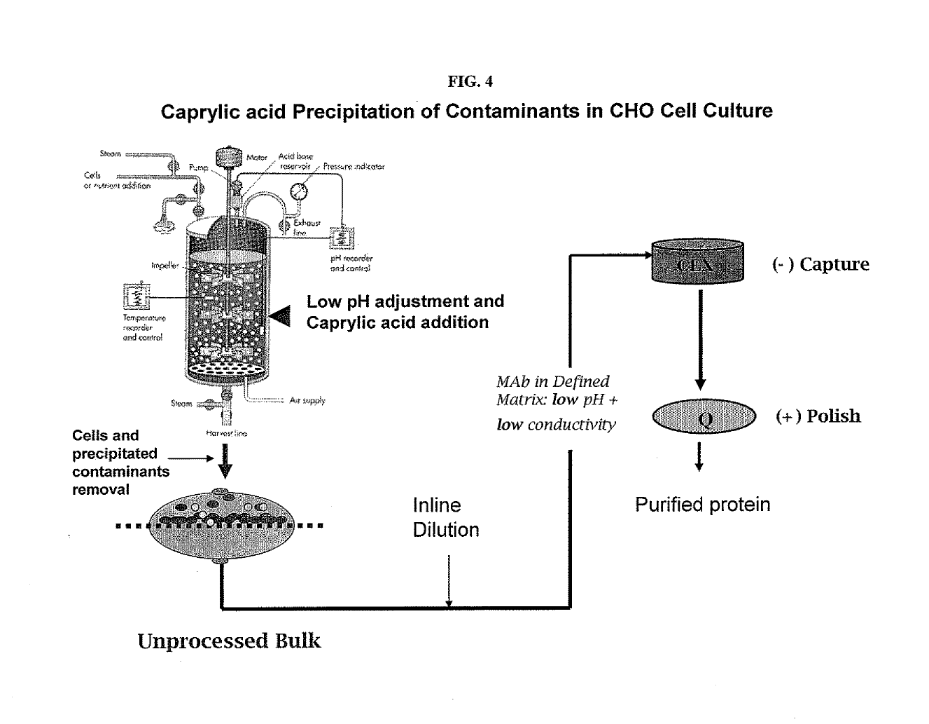

FIG. 5 shows the amount of contaminant precipitation (CHOP) and percentage recovery of secreted antibody (mAb recovery) at different concentrations of caprylic acid for a CHO cell line secreting a human monoclonal antibody.

FIG. 6 shows the amount of caprylic acid-mediated contaminant precipitation (CHOP) and percentage recovery of secreted antibody (mAb recovery) at various pH values for a CHO cell line secreting a human monoclonal antibody.

FIG. 7 shows the amount of contaminant precipitation (CHOP) and percentage recovery of secreted antibody (mAb recovery) at various times after the addition of caprylic acid for a CHO cell line secreting a human monoclonal antibody.

### DETAILED DESCRIPTION OF THE INVENTION

In certain aspects, the present invention provides a method of purifying a protein of interest from a mixture (e.g., cell culture, cell lysate or clarified bulk), which comprises adding caprylic acid to the mixture to precipitate the contaminants in the mixture, thereby removing them from the mixture.

As used herein the term “caprylic acid” refers to n-octanoic acid, or any derivatives thereof capable of selectively precipitating a contaminant when added to a solution.

As used herein, the term “protein of interest” is used in its broadest sense to include any protein (either natural or recombinant), present in a mixture, for which purification is desired. Such proteins of interest include, without limitation, hormones, growth factors, cytokines, immunoglobulins (e.g., antibodies), and immunoglobulin-like domain-containing molecules (e.g., ankyrin or fibronectin domain-containing molecules).

As used herein, a “cell culture” refers to cells in a liquid medium. Optionally, the cell culture is contained in a bioreactor. The cells in a cell culture can be from any organism including, for example, bacteria, fungus, mammals or plants. In a particular embodiment, the cells in a cell culture include cells transfected with an expression construct containing a nucleic acid that encodes a protein of interest (e.g., an antibody). Suitable liquid media include, for example, nutrient media and non-nutrient media. In a particular embodiment, the cell culture comprises a Chinese Hamster Ovary (CHO) cell line in nutrient media, not subject to purification by, for example, filtration or centrifugation.

As used herein, the term “clarified bulk” refers to a mixture from which particulate matter has been substantially removed. Clarified bulk includes cell culture, or cell lysate from which cells or cell debris has been substantially removed by, for example, filtration or centrifugation.

As used herein “bioreactor” takes its art recognized meaning and refers to a chamber designed for the controlled growth of a cell culture. The bioreactor can be of any size as long as it is useful for the culturing of cells, e.g., mammalian cells. Typically, the bioreactor will be at least 30 ml and may be at least 1, 10, 100, 250, 500, 1000, 2500, 5000, 8000, 10,000, 12,0000 liters or more, or any intermediate volume. The internal conditions of the bioreactor, including but not limited to pH and temperature, are typically controlled during the culturing period. A suitable bioreactor may be composed of (i.e., constructed of) any material that is suitable for holding cell cultures suspended in media under the culture conditions and is conducive to cell growth and viability, including glass, plastic or metal; the material(s) should not interfere with expression or stability of a protein of interest. One of ordinary skill in the art will be aware of, and will be able to choose, suitable bioreactors for use in practicing the present invention.

As used herein, a “mixture” comprises a protein of interest (for which purification is desired) and one or more contaminant, i.e., impurities. In one embodiment, the mixture is produced from a host cell or organism that expresses the protein of interest (either naturally or recombinantly). Such mixtures include, for example, cell cultures, cell lysates, and clarified bulk (e.g., clarified cell culture supernatant).

As used herein, the terms “separating” and “purifying” are used interchangeably, and refer to the selective removal of contaminants from a mixture containing a protein of interest (e.g., an antibody). The invention achieves this by precipitation of the contaminants using caprylic acid. Following precipitation, the contaminant precipitate can be removed from the mixture using any means compatible with the present invention, including common industrial methods such as centrifugation or filtration. This separation results in the recovery of a mixture with a substantially reduced level of contaminants, and thereby serves to increase the purity of the protein of interest (e.g., an antibody) in the mixture.

As used herein, the term “contaminant precipitate” refers to an insoluble substance comprising one or more contaminants formed in a solution due to the addition of a compound (e.g., caprylic acid) to the solution.

As used herein the term “contaminant” is used in its broadest sense to cover any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants.

In certain embodiments, the contaminant precipitate is separated from the cell culture using an art-recognized means, such as centrifugation, depth filtration and tangential flow filtration.

As used herein “depth filtration” is a filtration method that uses depth filters, which are typically characterized by their design to retain particles due to a range of pore sizes within a filter matrix. The depth filter's capacity is typically defined by the depth, e.g., 10 inch or 20 inch of the matrix and thus the holding capacity for solids. In a method of the present invention, depth filtration can be used to remove a contaminant precipitate from a mixture, including without limitation, a cell culture or clarified cell culture supernatant.

As used herein, the term “tangential flow filtration” refers to a filtration process in which the sample mixture circulates across the top of a membrane, while applied pressure causes certain solutes and small molecules to pass through the membrane. In a method of the present invention tangential flow filtration can be used to remove a contaminant precipitate from a mixture, including without limitation, a cell culture or clarified cell culture supernatant.

In certain aspects, methods of the present invention may be used to produce any protein of interest including, but not limited to, proteins having pharmaceutical, diagnostic, agricultural, and/or any of a variety of other properties that are useful in commercial, experimental or other applications. In addition, a protein of interest can be a protein therapeutic. In certain embodiments, proteins produced using methods of the present invention may be processed or modified. For example, a protein to be produced in accordance with the present invention may be glycosylated.

Thus, the present invention may be used to culture cells for production of any therapeutic protein, such as pharmaceutically or commercially relevant enzymes, receptors, receptor fusions, antibodies (e.g., monoclonal or polyclonal antibodies), antigen-binding fragments of an antibody, Fc fusion proteins, cytokines, hormones, regulatory factors, growth factors, coagulation/clotting factors, or antigen-binding agents. The above list of proteins is merely exemplary in nature, and is not intended to be a limiting recitation. One of ordinary skill in the art will know that other proteins can be produced in accordance with the present invention, and will be able to use methods disclosed herein to produce such proteins.

In one particular embodiment of the invention, the protein produced using the method of the invention is an antibody. The term “antibody” is used in the broadest sense to cover monoclonal antibodies (including full length monoclonal antibodies), polyclonal antibodies, multispecific antibodies (e.g., bispecific antibodies), antibody fragments, immunoadhesins and antibody-immunoadhesin chimerias.

An “antibody fragment” includes at least a portion of a full length antibody and typically an antigen binding or variable region thereof. Examples of antibody fragments include Fab, Fab′, F(ab′)₂, and Fv fragments; single-chain antibody molecules; diabodies; linear antibodies; and multispecific antibodies formed from engineered antibody fragments.

The term “monoclonal antibody” is used in the conventional sense to refer to an antibody obtained from a population of substantially homogeneous antibodies such that the individual antibodies comprising the population are identical except for possible naturally occurring mutations that may be present in minor amounts. Monoclonal antibodies are highly specific, being directed against a single antigenic site. This is in contrast with polyclonal antibody preparations which typically include varied antibodies directed against different determinants (epitopes) of an antigen, whereas monoclonal antibodies are directed against a single determinant on the antigen. The term “monoclonal”, in describing antibodies, indicates the character of the antibody as being obtained from a substantially homogeneous population of antibodies, and is not to be construed as requiring production of the antibody by any particular method. For example, monoclonal antibodies used in the present invention can be produced using conventional hybridoma technology first described by Kohler et al., Nature, 256:495 (1975), or they can be made using recombinant DNA methods (see, e.g., U.S. Pat. No. 4,816,567). Monoclonal antibodies can also be isolated from phage antibody libraries, e.g., using the techniques described in Clackson et al., Nature, 352:624-628 (1991); Marks et al., J. Mol. Biol., 222:581-597 (1991); and U.S. Pat. Nos. 5,223,409, 5,403,484, 5,571,698, 5,427,908, 5,580,717, 5,969,108, 6,172,197, 5,885,793, 6,521,404, 6,544,731, 6,555,313, 6,582,915 and 6,593,081).

The monoclonal antibodies described herein include “chimeric” and “humanized” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). “Humanized” forms of non-human (e.g., murine) antibodies are chimeric antibodies which contain minimal sequence derived from non-human immunoglobulin. For the most part, humanized antibodies are human immunoglobulins (recipient antibody) in which the hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature, 321:522-525 (1986); Riechmann et al., Nature, 332:323-329 (1988); and Presta, Curr. Op. Struct. Biol., 2:593-596 (1992).

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101, 5,585,089, 5,693,762 and 6,180,370 to Queen et al.).

The monoclonal antibodies described herein also include “human” antibodies, which can be isolated from various sources, including, e.g., from the blood of a human patient or recombinantly prepared using transgenic animals. Examples of such transgenic animals include KM-MOUSE® (Medarex, Inc., Princeton, N.J.) which has a human heavy chain transgene and a human light chain transchromosome (see WO 02/43478), XENOMOUSE® (Abgenix, Inc., Fremont Calif.; described in, e.g., U.S. Pat. Nos. 5,939,598, 6,075,181, 6,114,598, 6,150,584 and 6,162,963 to Kucherlapati et al.), and HUMAB-MOUSE® (Medarex, Inc.; described in, e.g., Taylor, L. et al., Nucleic Acids Research, 20:6287-6295 (1992); Chen, J. et al., International Immunology, 5:647-656 (1993); Tuaillon et al., Proc. Natl. Acad. Sci. USA, 90:3720-3724 (1993); Choi et al., Nature Genetics, 4:117-123 (1993); Chen, J. et al., EMBO J., 12: 821-830 (1993); Tuaillon et al., J. Immunol., 152:2912-2920 (1994); Taylor, L. et al., International Immunology, 6:579-591 (1994); and Fishwild, D. et al., Nature Biotechnology, 14:845-851 (1996); U.S. Pat. Nos. 5,545,806, 5,569,825, 5,625,126, 5,633,425, 5,789,650, 5,877,397, 5,661,016, 5,814,318, 5,874,299, 5,770,429 and 5,545,807; and PCT Publication Nos. WO 92/03918, WO 93/12227, WO 94/25585, WO 97/13852, WO 98/24884 and WO 99/45962, and WO 01/14424 to Korman et al.). Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Mixtures Containing a Protein of Interest

The methods of the invention can be applied to any mixture containing a protein of interest. In one embodiment, the mixture is obtained from or produced by living cells that express the protein to be purified (e.g., naturally or by genetic engineering). Optionally, the cells in a cell culture include cells transfected with an expression construct containing a nucleic acid that encodes a protein of interest. Methods of genetically engineering cells to produce proteins are well known in the art. See e.g., Ausubel et al., eds., Current Protocols in Molecular Biology, Wiley, New York (1990) and U.S. Pat. Nos. 5,534,615 and 4,816,567, each of which are specifically incorporated herein by reference. Such methods include introducing nucleic acids that encode and allow expression of the protein into living host cells. These host cells can be bacterial cells, fungal cells, or, preferably, animal cells grown in culture. Bacterial host cells include, but are not limited to E. coli cells. Examples of suitable E. coli strains include: HB101, DHSα, GM2929, JM109, KW251, NM538, NM539, and any E. coli strain that fails to cleave foreign DNA. Fungal host cells that can be used include, but are not limited to, Saccharomyces cerevisiae, Pichia pastoris and Aspergillus cells.

A number of mammalian cell lines are suitable host cells for expression of proteins of interest. Mammalian host cell lines include, for example, COS, PER.C6, TM4, VERO076, DXB11, MDCK, BRL-3A, W138, Hep G2, MMT, MRC 5, FS4, CHO, 293T, A431, 3T3, CV-1, C3H10T1/2, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NS0) and C2C12 cells, as well as transformed primate cell lines, hybridomas, normal diploid cells, and cell strains derived from in vitro culture of primary tissue and primary explants. New animal cell lines can be established using methods well known by those skilled in the art (e.g., by transformation, viral infection, and/or selection). Any eukaryotic cell that is capable of expressing the protein of interest may be used in the disclosed cell culture methods. Numerous cell lines are available from commercial sources such as the American Type Culture Collection (ATCC). In one embodiment of the invention, the cell culture, e.g., the large-scale cell culture, employs hybridoma cells. The construction of antibody-producing hybridoma cells is well known in the art. In one embodiment of the invention, the cell culture, e.g., the large-scale cell culture, employs CHO cells to produce the protein of interest such as an antibody (see, e.g., WO 94/11026). Various types of CHO cells are known in the art, e.g., CHO-K1, CHO-DG44, CHO-DXB11, CHO/dhfr⁻ and CHO-S.

In certain embodiments, the present invention contemplates, prior to purifying a protein of interest from a cell culture, monitoring particular conditions of the growing cell culture. Monitoring cell culture conditions allows for determining whether the cell culture is producing the protein of interest at adequate levels. For example, small aliquots of the culture are periodically removed for analysis in order to monitor certain cell culture conditions. Cell culture conditions to be monitored include, but not limited to, temperature, pH, cell density, cell viability, integrated viable cell density, lactate levels, ammonium levels, osmolality, and titer of the expressed protein. Numerous techniques are well known to those of skill in the art for measuring such conditions/criteria. For example, cell density may be measured using a hemocytometer, an automated cell-counting device (e.g., a COULTER COUNTER®, Beckman Coulter Inc., Fullerton, Calif.), or cell-density examination (e.g., CEDEX®, Innovatis, Malvern, Pa.). Viable cell density may be determined by staining a culture sample with Trypan blue. Lactate and ammonium levels may be measured, e.g., with the BIOPROFILE® 400 Chemistry Analyzer (Nova Biomedical, Waltham, Mass.), which takes real-time, online measurements of key nutrients, metabolites, and gases in cell culture media. Osmolality of the cell culture may be measured by, e.g., a freezing point osmometer. HPLC can be used to determine, e.g., the levels of lactate, ammonium, or the expressed protein. In one embodiment of the invention, the levels of expressed protein can be determined by using, e.g., protein A HPLC. Alternatively, the level of the expressed protein can be determined by standard techniques such as Coomassie staining of SDS-PAGE gels, Western blotting, Bradford assays, Lowry assays, biuret assays, and UV absorbance. Optionally, the present invention may include monitoring the post-translational modifications of the expressed protein, including phosphorylation and glycosylation.

In a specific embodiment, methods of the present invention comprise effectively removing contaminants from a mixture (e.g., a cell culture, cell lysate or clarified bulk) which contains a high concentration of a protein of interest (e.g., an antibody). For example, the concentration of a protein of interest may range from about 0.5 to about 50 mg/ml (e.g., 0.5, 1, 5, 10, 15, 20, 25, 30, 35, 40, 45 or 50 mg/ml).

Preparation of Mixtures Initially Depends on the Manner of Expression of the protein. Some cell systems directly secrete the protein (e.g., an antibody) from the cell into the surrounding growth media, while other systems retain the antibody intracellularly. For proteins produced intracellularly, the cell can be disrupted using any of a variety of methods, such as mechanical shear, osmotic shock, and enzymatic treatment. The disruption releases the entire contents of the cell into the homogenate, and in addition produces subcellular fragments which can be removed by centrifugation or by filtration. A similar problem arises, although to a lesser extent, with directly secreted proteins due to the natural death of cells and release of intracellular host cell proteins during the course of the protein production run.

In one embodiment, cells or cellular debris are removed from the mixture, for example, to prepare clarified bulk. The methods of the invention can employ any suitable methodology to remove cells or cellular debris. If the protein is produced intracellularly, as a first step, the particulate debris, either host cells or lysed fragments, can be removed, for example, by a centrifugation or filtration step in order to prepare a mixture which is then subjected to purification according the methods described herein (i.e., from which a protein of interest is purified). If the protein is secreted into the medium, the recombinant host cells may be separated from the cell culture medium by, e.g., centrifugation, tangential flow filtration or depth filtration, in order to prepare a mixture from which a protein of interest is purified.

In another embodiment, cell culture or cell lysate is used directly without first removing the host cells. Indeed, the methods of the invention are particularly well suited to using mixtures comprising a secreted protein and a suspension of host cells.

### Contaminants Precipitation by Caprylic Acid

According to the present invention, removal of contaminants from a mixture, (e.g., cell culture, cell lysates or clarified bulk) is achieved by precipitation with caprylic acid. Such methods are particularly advantageous in that they can achieve the removal of 60% or more (e.g., more than 65, 70, 75, 80, 85, 90, 95, or 99%) of protein contaminants (e.g., host cell proteins) from cell culture or clarified bulk. Accordingly, after precipitation of contaminants with caprylic acid, cell culture and clarified bulk can contain less than about 10000 ng/mg (e.g., less than about 10000, 5000, 1000, 500, 200, 100, 50, 25, or 10 ng/mg) of protein contaminants (e.g., host cell proteins).

The methods of the invention are also useful for reducing the nucleic acid (e.g., DNA) content of a mixture (e.g., cell culture, cell lysates, and clarified bulk). For example, after precipitation of contaminants with caprylic acid, mixtures (e.g., cell culture, cell lysates, or clarified bulk) can have a DNA content of less than about 500 pg/mg (e.g., less than about 500, 100, 50, 10, 5, 1 or 0.5 pg/mg). In a particular embodiment, the concentration of DNA contaminants in the mixture is reduced by about one million fold to be less than 5 pg/mg of protein.

The concentration of caprylic acid sufficient to precipitate contaminants from a particular mixture can be determined empirically for each protein mixture using methods described herein. The final concentration of caprylic acid added to the mixture is typically between about 0.05% and 5% volume/volume (v/v), preferably between about 0.5% and 2% (v/v) (e.g., about 1.0%).

In certain embodiments, the pH of the mixture is altered to facilitate precipitation. The optimum pH required to facilitate caprylic acid precipitation of a particular contaminant can be determined empirically for each protein mixture using methods described herein. Preferably the final pH of the mixture is altered to be between about 3 and 8 (e.g., about 4, 5, 6, or 7). In a particular embodiment, the pH of the mixture is altered to be about 4.5 or less. The pH of the mixture can be adjusted before or after the addition of caprylic acid to the mixture. In a preferred embodiment, the pH of the mixture is adjusted before the addition of caprylic acid. In general, any art recognized acids or buffers can be used to alter the pH of a mixture, including, for example, acetate- and citrate-containing buffers. An advantage of using a bioreactor cell culture is that the pH of the cell culture medium can be monitored and adjusted by addition of one or more suitable acids or buffers to the cell culture medium in the bioreactor.

In certain embodiments, the caprylic acid is added to the mixture and mixed for a particular length of time prior to removing the contaminant precipitate. The optimum length of mixing required to facilitate caprylic acid precipitation of a particular contaminant can be determined empirically for each protein mixture using methods described herein. Preferably the mixing time is greater than about 30 minutes (e.g., about 60, 90, 120, 240, or 480 minutes). In a particular embodiment, the mixing time is about 60 minutes.

The methods of invention are particularly well suited to purifying secreted proteins (e.g., antibodies) from cell culture or cell lysate. In a particular embodiment, caprylic acid is added directly to a cell culture without first removing the cells, or cellular debris. After formation of a contaminant precipitate, both the contaminant precipitate and the cells are removed from the mixture in a single step using an art recognized separation technique (e.g., centrifugation, tangential flow filtration or depth filtration). This method is particularly advantageous since it replaces several steps commonly used in antibody manufacturing, and results in the rapid and cost effective production of a protein mixture with a significant reduction of contaminants and suitable for downstream purification processes such as chromatography.

### Further Purification of the Protein

Following the removal of the contaminant precipitate and/or cells, the mixture (e.g., an antibody-containing cell culture sample) has a greatly reduced level of contaminants (e.g., host cell DNA and proteins) and can be directly used in chromatography for the further purification of the protein (e.g., an antibody). Any suitable art recognized chromatography technique can be employed to further purify the protein including, without limitation, ion-exchange, HIC, affinity (e.g., Protein A), mimetic, and mixed mode. Suitable chromatography methods are described, for example, in WO 06/110277, the entire contents of which are hereby incorporated by reference herein. The pH, conductivity or protein concentration of the mixture can be adjusted to as necessary for the particular chromatography application.

As used herein the term “chromatography” refers to the process by which a solute of interest, e.g., a protein of interest, in a mixture is separated from other solutes in the mixture by percolation of the mixture through an adsorbent, which adsorbs or retains a solute more or less strongly due to properties of the solute, such as pI, hydrophobicity, size and structure, under particular buffering conditions of the process. In a method of the present invention, chromatography can be used to remove contaminants after the precipitate is removed from a mixture, including without limitation, a cell culture or clarified cell culture supernatant.

As used herein, the term “hydrophobic charge induction chromatography” (or “HCIC”) is a type of mixed mode chromatographic process in which the protein of interest in the mixture binds to a dual mode (i.e., there is one mode for binding and another mode for elution), ionizable ligand [see Boschetti et al., Genetic Engineering News, 20(13) (2000)] through mild hydrophobic interactions in the absence of added salts (e.g., a lyotropic salts). A “hydrophobic charge induction chromatography resin” is a solid phase that contains a ligand which has the combined properties of thiophilic effect (i.e., utilizing the properties of thiophilic chromatography), hydrophobicity and an ionizable group for its separation capability. Thus, an HCIC resin used in a method of the invention contains a ligand that is ionizable and mildly hydrophobic at neutral (physiological) or slightly acidic pH, e.g., about pH 5 to 10, preferably about pH 6 to 9.5. At this pH range, the ligand is predominantly uncharged and binds a protein of interest via mild non-specific hydrophobic interaction. As pH is reduced, the ligand acquires charge and hydrophobic binding is disrupted by electrostatic charge repulsion towards the solute due to the pH shift. Examples of suitable ligands for use in HCIC include any ionizable aromatic or heterocyclic structure (e.g., those having a pyridine structure, such as 2-aminomethylpyridine, 3-aminomethylpyridine and 4-aminomethylpyridine, 2-mercaptopyridine, 4-mercaptopyridine or 4-mercaptoethylpyridine, mercaptoacids, mercaptoalcohols, imidazolyl based, mercaptomethylimidazole, 2-mercaptobenzimidazole, aminomethylbenzimidazole, histamine, mercaptobenzimidazole, diethylaminopropylamine, aminopropylmorpholine, aminopropylimidazole, aminocaproic acid, nitrohydroxybenzoic acid, nitrotyrosine/ethanolamine, dichlorosalicylic acid, dibromotyramine, chlorohydroxyphenylacetic acid, hydroxyphenylacetic acid, tyramine, thiophenol, glutathione, bisulphate, and dyes, including derivatives thereof; see Burton et al., Journal of Chromatography A, 814:81-81 (1998) and Boschetti, Journal of Biochemical and Biophysical Methods, 49:361-389 (2001), which are hereby incorporated by reference in their entireties), which has an aliphatic chain and at least one sulfur atom on the linker arm and/or ligand structure. An example of an HCIC resin includes MEP HyperCel (Pall Corporation; East Hills, N.Y.).

The terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange, anion exchange, and mixed mode chromatographies.

The phrase “ion exchange material” refers to a solid phase that is negatively charged (i.e., a cation exchange resin) or positively charged (i.e., an anion exchange resin). In one embodiment, the charge can be provided by attaching one or more charged ligands (or adsorbents) to the solid phase, e.g., by covalent linking. Alternatively, or in addition, the charge can be an inherent property of the solid phase (e.g., as is the case for silica, which has an overall negative charge).

A “cation exchange resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MONO S®, MINI S®, Source 15S and 30S, SP SEPHAROSE® Fast Flow, SP SEPHAROSE® High Performance from GE Healthcare, TOYOPEARL® SP-650S and SP-650M from Tosoh, MACRO-PREP® High S from Bio-Rad, Ceramic HyperD S, TRISACRYL® M and LS SP and Spherodex LS SP from Pall Technologies); a sulfoethyl based group (e.g., FRACTOGEL® SE, from EMD, POROS® S-10 and S-20 from Applied Biosystems); a sulphopropyl based group (e.g., TSKgel SP 5PW and SP-5PW-HR from Tosoh, POROS® HS-20 and HS 50 from Applied Biosystems); a sulfoisobutyl based group (e.g., FRACTOGEL® EMD SO₃⁻ from EMD); a sulfoxyethyl based group (e.g., SE52, SE53 and Express-Ion S from Whatman), a carboxymethyl based group (e.g., CM SEPHAROSE® Fast Flow from GE Healthcare, Hydrocell CM from BioChrom Labs Inc., MACRO-PREP® CM from Bio-Rad, Ceramic HyperD CM, TRISACRYL® M CM, TRISACRYL® LS CM, from Pall Technologies, Matrex CELLUFINE® C500 and C200 from Millipore, CM52, CM32, CM23 and Express-Ion C from Whatman, TOYOPEARL® CM-650S, CM-650M and CM-650C from Tosoh); sulfonic and carboxylic acid based groups (e.g., BAKERBOND® Carboxy-Sulfon from J. T. Baker); a carboxylic acid based group (e.g., WP CBX from J. T Baker, DOWEX® MAC-3 from Dow Liquid Separations, AMBERLITE® Weak Cation Exchangers, DOWEX® Weak Cation Exchanger, and DIAION® Weak Cation Exchangers from Sigma-Aldrich and FRACTOGEL® EMD COO- from EMD); a sulfonic acid based group (e.g., Hydrocell SP from BioChrom Labs Inc., DOWEX® Fine Mesh Strong Acid Cation Resin from Dow Liquid Separations, UNOsphere S, WP Sulfonic from J. T. Baker, SARTOBIND® S membrane from Sartorius, AMBERLITE® Strong Cation Exchangers, DOWEX® Strong Cation and DIAION® Strong Cation Exchanger from Sigma-Aldrich); and a orthophosphate based group (e.g., P11 from Whatman).

An “anion exchange resin” refers to a solid phase which is positively charged, thus having one or more positively charged ligands attached thereto. Any positively charged ligand attached to the solid phase suitable to form the anionic exchange resin can be used, such as quaternary amino groups Commercially available anion exchange resins include DEAE cellulose, POROS® PI 20, PI 50, HQ 10, HQ 20, HQ 50, D 50 from Applied Biosystems, SARTOBIND® Q from Sartorius, MONO Q®, MINI Q®, Source 15Q and 30Q, Q, DEAE and ANX SEPHAROSE® Fast Flow, Q SEPHAROSE® high Performance, QAE SEPHADEX® and FAST Q SEPHAROSE® (GE Healthcare), WP PEI, WP DEAM, WP QUAT from J. T. Baker, Hydrocell DEAE and Hydrocell QA from BioChrom Labs Inc., UNOsphere Q, MACRO-PREP® DEAE and MACRO-PREP® High Q from Bio-Rad, Ceramic HyperD Q, ceramic HyperD DEAE, TRISACRYL® M and LS DEAE, Spherodex LS DEAE, QMA SPHEROSIL® LS, QMA SPHEROSIL® M and MUSTANG® Q from Pall Technologies, DOWEX® Fine Mesh Strong Base Type I and Type II Anion Resins and DOWEX® MONOSPHERE® 77, weak base anion from Dow Liquid Separations, INTERCEPT® Q membrane, Matrex CELLUFINE® A200, A500, Q500, and Q800, from Millipore, FRACTOGEL® EMD TMAE, FRACTOGEL® EMD DEAE and FRACTOGEL® EMD DMAE from EMD, AMBERLITE® weak strong anion exchangers type I and II, DOWEX® weak and strong anion exchangers type I and II, DIAION® weak and strong anion exchangers type I and II, DUOLITE® from Sigma-Aldrich, TSKgel Q and DEAE 5PW and 5PW-HR, TOYOPEARL® SuperQ-650S, 650M and 650C, QAE-550C and 650S, DEAE-650M and 650C from Tosoh, QA52, DE23, DE32, DE51, DE52, DE53, Express-Ion D and Express-Ion Q from Whatman.

A “mixed mode ion exchange resin” or “mixed mode” refers to a solid phase which is covalently modified with cationic, anionic, and/or hydrophobic moieties. Examples of mixed mode ion exchange resins include BAKERBOND® ABX (J. T. Baker, Phillipsburg, N.J.), ceramic hydroxyapatite type I and II and fluoride hydroxyapatite (Bio-Rad, Hercules, Calif.) and MEP and MBI HyperCel (Pall Corporation, East Hills, N.Y.).

The present disclosure is further illustrated by the following examples, which should not be construed as further limiting. The contents of all figures and all references, patents and published patent applications cited throughout this application are expressly incorporated herein by reference in their entireties.

### Example 1

### Contaminant Precipitation Using Caprylic Acid

This example demonstrates the effectiveness of caprylic acid precipitation at removing host cell proteins from cell culture or clarified bulk (CB) samples. Optimal precipitation conditions for a particular sample can be determined empirically by varying the caprylic acid concentration, pH and mixing time and determining the antibody and host cell protein level remaining in the supernatant after caprylic acid-induced precipitation.

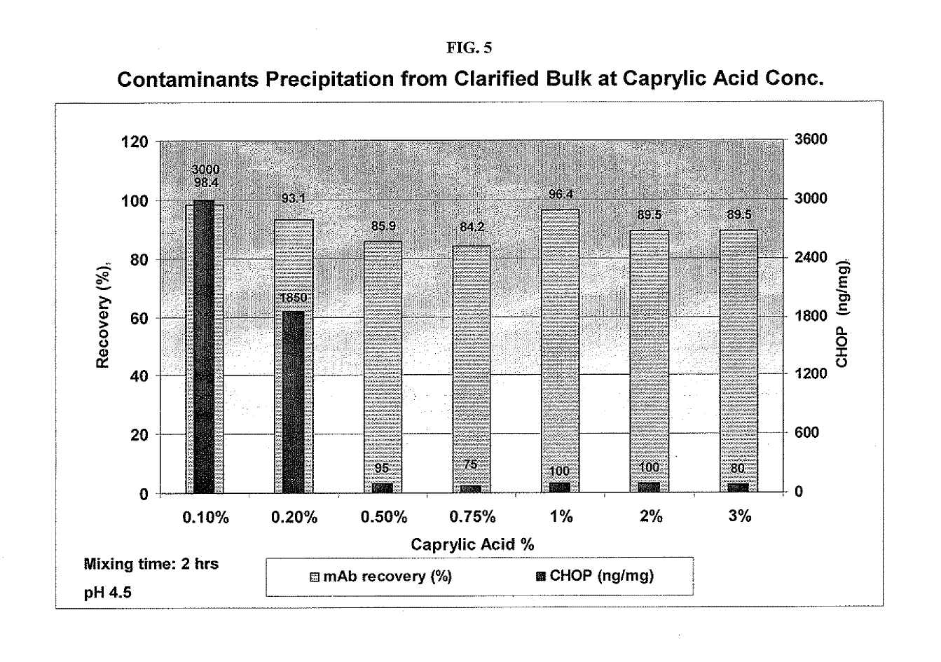

To determine the optimal caprylic acid concentration for selective precipitation of contaminants, different final concentrations of caprylic acid were added after pH was adjusted to 4.5 to a series of identical clarified bulk samples comprising antibody-expressing CHO cells. The samples were mixed continuously for 2 hours to form a precipitate. The precipitate was then removed and the amount of remaining antibody and host cell protein quantified. Representative precipitation curves are depicted in FIG. 5 and show that greater than 0.2% caprylic acid was required for maximal precipitation of host protein from the clarified bulk sample tested.

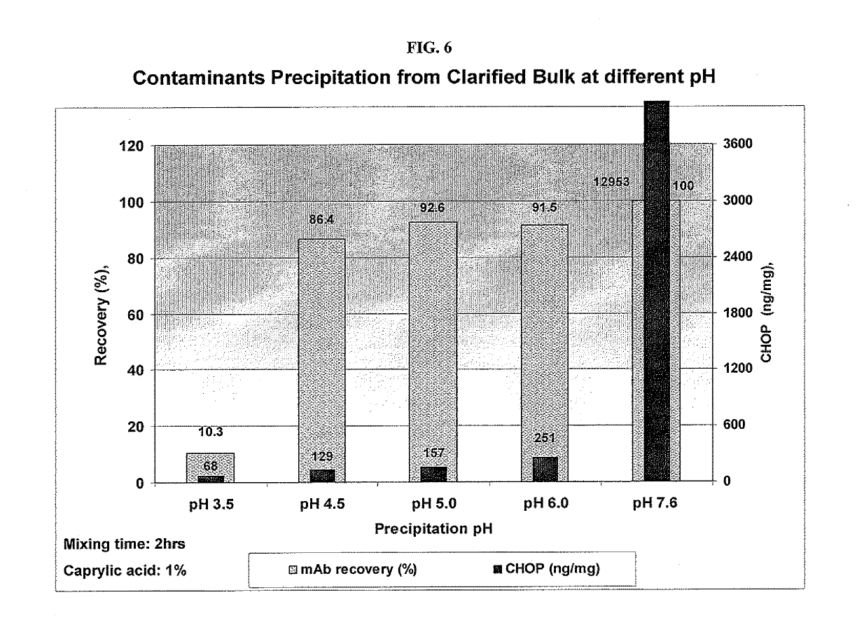

To determine the optimal pH for selective precipitation of contaminants by caprylic acid, the pH was adjusted first and 1% caprylic acid was added to a series of clarified bulk samples comprising antibody-expressing CHO cells. The samples were mixed continuously for 2 hours to form a precipitate. The precipitate was then removed and the amount of remaining antibody and host cell protein quantified. Representative precipitation curves are depicted in FIG. 6 and show that a pH of greater than 3.5, 1% caprylic acid was required for selective precipitation of host cell protein from the clarified bulk sample tested.

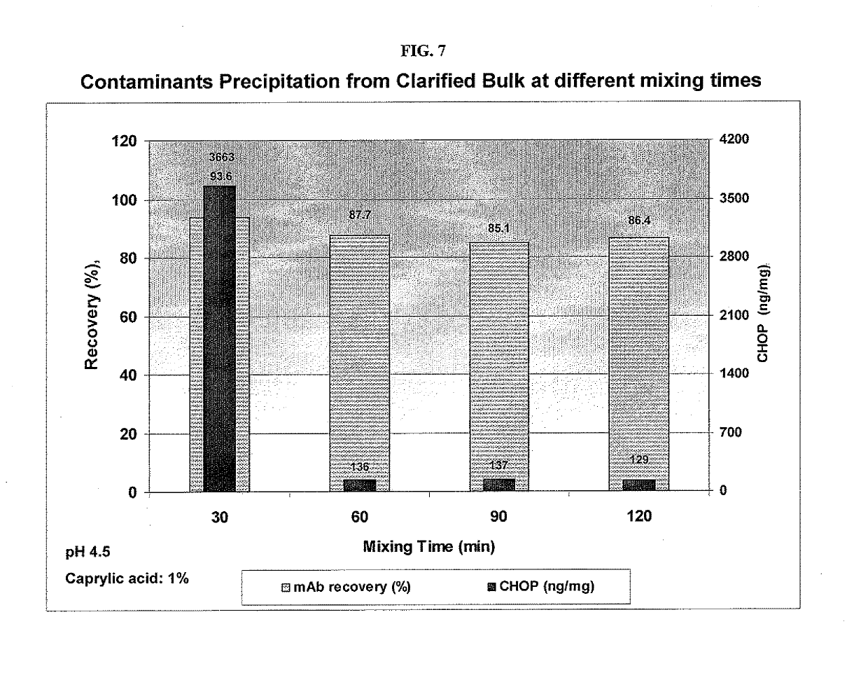

To determine the optimal mixing time for selective precipitation of contaminants by caprylic acid, the pH of the sample was adjusted to 4.5 and 1% caprylic acid was added to a series of identical clarified bulk samples comprising antibody-expressing CHO cells. The samples were mixed continuously for various lengths of time to form a precipitate. The precipitate was then removed and the amount of remaining antibody and host cell protein quantified. Representative precipitation curves are depicted in FIG. 7 and show that greater than 30 minutes of mixing was required after the addition of caprylic acid for maximal precipitation of host protein from the clarified bulk sample tested.

Table 1 provides data illustrating the effective removal of CHO cell proteins from two clarified CHO cell culture supernatants containing high concentrations of a human monoclonal antibody, using caprylic acid precipitation. For both cell culture supernatants, the precipitation step resulted in only a minor (1-2%) loss of antibody and about a 600-fold decrease in host cell protein contaminants.

TABLE 1
Caprylic acid precipitation from clarified CHO cell culture
supernatants containing antibody at >10 mg/mL.

| | Load CHOP | Post treatment CHOP | Recovery |
|---|---|---|---|
| CB | (ng/mg) | (ng/mg) | (%) |
| CB 1 | ~3 × 10⁴ | <50 | 99 |
| CB 2 | ~3 × 10⁴ | <50 | 98 |

Caprylic acid can also be used to remove host cell contaminants directly from cell culture samples containing an antibody and antibody-secreting host cells. In this case, caprylic acid is added directly to a cell culture after the pH of the cell culture is adjusted to optimize precipitation of contaminants by caprylic acid. Table 2 shows the results of experiments in which two cell culture samples containing human monoclonal antibodies (Humab-1 and 2) were treated with 1% caprylic acid, at pH 4.5 for 2 hours. In both cases, the amount of CHO host cell protein (CHOP) was reduced by over 1000-fold and the recovery of the Humab was over 80%. The amount of antibody lost in this purification is less than that lost cumulatively in the conventional clarification, concentration and diafiltration TFF steps (percentages in parenthesis).

TABLE 2
Caprylic acid precipitation of contaminants from CHO cell culture.

| | | CHOP post | |
|---|---|---|---|
| | CHOP in | Caprylic Acid | |
| | Cell Culture | Precipitation | Step recovery |
| | supernatant | of cell culture | for precipitation |
| Product | (ng/mg) | (ng/mg) | (%) |
| Humab-1 | 27499 | 161 | 87.8 (~20%) |
| ~12 × 10⁶ | | | |
| Cells/ml | | | |
| Humab-2 | 32189 | 12 | 83.5 (~20 to 25%) |
| ~120 × 10⁶ | | | |
| Cells/ml | | | |

% in parenthesis indicates cumulative product loss in conventional clarification and concentration and diafiltration TFF steps.

### Example 2

### Integration of Caprylic Acid Precipitation with Cation-Exchange Chromatography

This example demonstrates the compatibility of mixtures purified using caprylic acid precipitation for direct use in downstream chromatography steps. A CHO cell culture was treated with caprylic acid to precipitate host cell contaminants and the resultant contaminant precipitate was removed. The caprylic acid-treated mixture was then subject to CEX using two different high-capacity CEX resins. As shown in Table 3, for both CEX resins, the final, purified antibody had a purity greater than 99% and a CHOP content of less than 10 ng/mg of antibody.

Table 3. Integration of Caprylic Acid Precipitation with Cation-Exchange Chromatography.

90-100 mg/mL resin binding capacity was achieved. Caprylic acid-treated cell culture mixture diluted prior to column loading.

| | Load CHOP | Elution CHOP | Elution purity | Recovery |
|---|---|---|---|---|
| Resin | (ng/mg) | (ng/mg) | (%) | (%) |
| GigaCap S | ~100 | <10 | 99.6 | 94 |
| GigaCap CM | ~100 | <10 | 99.8 | 95 |

### EQUIVALENTS

Those skilled in the art will recognize or be able to ascertain using no more than routine experimentation, many equivalents of the specific embodiments of the invention described herein. Such equivalents are intended to be encompassed by the following claims.

### INCORPORATION BY REFERENCE

All patents, pending patent applications, and other publications cited herein are hereby incorporated by reference in their entireties.

### REFERENCES

Ahamed, T. et al., “Selection of pH-related parameters in ion-exchange chromatography using pH-gradient operations”, Journal of Chromatography A, 1194(1):22-29 (2007). Available online Dec. 8, 2007.
Arunakumari, A. et al., “Alternatives to Protein A: Improved Downstream Process Design for Human Monoclonal Antibody Production”, Biopharm International (Feb. 2, 2007).
Foster P. R. et al., “The kinetics of protein salting-out: precipitation of yeast enzymes by ammonium sulfate”, Biotechnology and Bioengineering, 18(4):545-580 (2004).
Gagnon, P., “Purification Tools for Monoclonal Antibodies”, Validated Biosystems, Tucson, Ariz. (1996) (ISBN: 0-9653515-9-9).
Gagnon, P., “Use of Hydrophobic Interaction Chromatography with a Non-Salt Buffer System for Improving Process Economics in Purification of Monoclonal Antibodies”, Waterside Conference on Monoclonal and Recombinant
Antibodies, Miami Fla., Tosoh (2000).
Matheus, S. et al., “Liquid high concentration IgG1 antibody formulations bt precipitation”, Journal of Pharmaceutical Sciences (2008) (Epub ahead of print).
Moscariello, J., “Comparison of Potential Monoclonal Antibody
Purification Processes with Two Chromatography Steps”, BioProcess International Conference, Anaheim, Calif. (2008).
Shields, C., “Advances in Single Use Capture Chromatography”, BioProcess International Conference, Anaheim, Calif. (2008).
Ramanan, S. et al., “Method of Isolating Antibodies by Precipitation”, WO 2008/100578 A2 (2008).
Vilmorin, P., “Scale-up evaluation of selective antibody precipitation and continuous recovery with a disc-stack centrifuge”, BioProcess International Conference, Anaheim, Calif. (2008).
Wang, J. et al., “Optimizing the primary recovery step in nonaffinity purification schemes for HuMAbs”. BioPharm International (Mar. 2, 2008).
Zellner et al., “Quantitative validation of different protein precipitation methods in proteome analysis of blood platelets”, Electrophoresis, 26(12):2481-2489 (2005).

1. A method of purifying a protein of interest from a cell culture comprising the protein of interest and one or more contaminants, comprising:
a) adding caprylic acid to the cell culture to form a contaminant precipitate; and
b) separating the contaminant precipitate from the cell culture, thereby purifying the protein of interest.

2. The method of claim 1, wherein the cell culture is in a bioreactor.

3. The method of claim 1, wherein cells in the cell culture are lysed prior to the addition of caprylic acid.

4. The method of claim 1, wherein cells or cellular debris are removed from the cell culture prior to the addition of caprylic acid.

5. The method of claim 1, further comprising the step of subjecting the purified protein to a chromatography selected from the group consisting of ion exchange, hydrophobic interaction, affinity, mimetic, and mixed mode.

6. The method of claim 1, wherein the pH of the mixture is adjusted prior to the addition of caprylic acid.

7. The method of claim 1, wherein at least 60% of the contaminants are removed.

8. The method of claim 1, wherein the protein of interest in the cell culture has a high concentration before purification.

9. The method of claim 1, wherein at least about 60% of the protein of interest remains in the cell culture fluid after separation from the contaminant precipitate.

10. The method claim 1, wherein the cell culture is a mammalian cell culture.

11. The method of claim 10, wherein the cell culture is a Chinese Hamster Ovary (CHO) cell culture.

12. The method claim 1, wherein the protein of interest is an antibody.

13. The method of claim 12, wherein the antibody is a monoclonal antibody.

14. The method of claim 13, wherein the monoclonal antibody is selected from the group consisting of a human, humanized and chimeric antibody.

15. A method for removing contaminants from a mixture containing a protein of interest and one or more contaminants, comprising:
a) adjusting the pH of the mixture to less than 5;
b) adding a sufficient concentration of caprylic acid to the mixture to form a contaminant precipitate; and
c) separating the contaminant precipitate from the mixture, thereby removing contaminants from a mixture.

16. The method of claim 1, wherein the contaminant precipitate is separated from the cell culture by centrifugation, depth filtration or tangential flow filtration.

17. The method of claim 1, wherein the pH of the cell culture is between about 3 and 8.

18. The method of claim 1, wherein the pH of the mixture is less than 5.

19. The method of claim 1, wherein the contaminants comprise molecules selected from host cell proteins and nucleic acids.

20. The method of claim 1, wherein the final concentration of the caprylic acid is between about 0.05 and 5% (v/v).
